(19) United States
(12) Patent Application Publication
(10) Pub. No.: US 2022/0152087 A1
(43) Pub. Date: May 19, 2022

(54) CYCLODEXTRIN DERIVATIVES REDUCING FLAVIVIRUS NS1-INDUCED ENDOTHELIAL HYPERPERMEABILITY AND VASCULAR LEAK

(71) Applicant: CYCLOLAB CYCLODEXTRIN RESEARCH AND DEVELOPMENT LABORATORY LTD., Budapest (HU)

(72) Inventors: Tamas Sohajda, Halasztelek (HU); Istvan Puskas, Budapest (HU); Eva Harris, Berkeley, CA (US); Francielle Tramontini Gomes de Sousa, Berkeley, CA (US); Robert Beatty, Berkeley, CA (US); Peter Laing, Cambridge (GB)

(73) Assignee: CYCLOLAB CYCLODEXTRIN RESEARCH AND DEVELOPMENT LABORATORY LTD., Budapest (HU)

(21) Appl. No.: 17/097,977

(22) Filed: Nov. 13, 2020

(51) Int. Cl. A61K 31/724; A61P 9/10

(52) U.S. Cl. CPC A61K 31/724; A61P 9/10

(57) ABSTRACT

The present invention generally relates to cyclodextrin derivatives, including Dexolve®, sulfobutylether beta cyclodextrin interfering with the activity of the viral nonstructural protein NS1 produced by a flavivirus. More particularly, the present invention relates to method of treatment using cyclodextrin compounds for inhibiting or treating a condition resulting from a flavivirus infection, particularly vascular leakage condition and increased virus dissemination.

### FIELD OF THE INVENTION

The present invention generally relates to cyclodextrin derivatives, including Dexolve® sulfobutylether beta cyclodextrin, interfering with the activity of the viral nonstructural protein 1 (NS1) produced by flaviviruses or members of the Flaviviridae family that produce NS1 protein. More particularly, the present invention relates to method of treatment using cyclodextrin compounds as therapy for preventing or treating a condition resulting from a flavivirus infection, particularly endothelial permeability leading to vascular leakage and/or virus dissemination.

### BACKGROUND OF THE INVENTION

Cyclodextrins (CD) are a group of cyclic oligosaccharides that are obtained from the enzymatic transformation of starch by the action of the enzyme cyclodextrin glycosyltransferase elaborated by e.g. bacterium Bacillus macerans. Various methods exist for producing cyclodextrin glycosyltransferase as well as for making and isolating cyclodextrins. Cyclodextrins usually contain six to eight alpha-D-glucopyranose units linked at the 1,4 positions by alpha linkages, as in amylose. The molecules containing six, seven, or eight alpha-D-glucopyranose units are commonly known as alpha-cyclodextrin or cyclohexaamylose, beta-cyclodextrin or cycloheptaamylose, and gamma-cyclodextrin or cyclooctaamylose, respectively. When reference is made here to “cyclodextrin”, it is intended to include the foregoing forms of cyclodextrin as well as molecules where the number of oligomerizations is over 8. As a consequence of the cyclic arrangement, cyclodextrins are characterized as having neither a reducing end group nor a non-reducing end group. The cyclic arrangement and the conformation of the alpha-D-glucopyranose units limits the free rotation around the glycosidic bonds, resulting in conical shaped molecules with the primary hydroxyl groups situated at the small end of the cone and the secondary hydroxyls situated at the large opening to the cone. The cavity is lined by hydrogen atoms from C3 and C5 along with the glucosidic oxygen atoms, resulting in a relatively lipophilic cavity but hydrophilic outer surface.

As a result of the two separate regions of different polarity and the changes in solvent structure that occur upon complex formation, cyclodextrins can form complexes with a variety of organic molecules or hydrophobic moieties of macromolecules. The formation of cyclodextrin inclusion complexes with molecules is referred to as the host-guest phenomenon.

These unique properties of cyclodextrins have resulted in their commercial application in agriculture, water treatment, household products, and drug delivery systems. The application of cyclodextrins in the pharmaceutical field has resulted in time-release microencapsulation, improved stability, and increased aqueous solubility of various drugs. Apart from the drug delivery properties of cyclodextrins, the curative pharmacological properties of cyclodextrins are also being explored. Some cyclodextrins of therapeutic value have been approved as active pharmaceutical ingredients such as Sugammadex (used in anesthesia) and 2-hydroxypropyl-beta-cyclodextrin (applied for Niemann Pick C disease treatment). Severe dengue virus (DENV) infections are characterized by increased vascular permeability and hemorrhagic manifestations. DENV has 4 serotypes, DENV-1 to DENV-4. Despite substantial morbidity and mortality, no therapeutic agents exist for treatment of dengue, and the currently available vaccine does not confer full protection. Thus, development of therapeutic and/or preventive drugs is urgently needed. Nonstructural protein 1 (NS1) plays important roles in host immune evasion and viral pathogenesis, for instance, by directly triggering endothelial barrier dysfunction and inducing inflammatory responses, contributing to vascular leak in vivo. It was previously shown that the endothelial disruption observed with all serotypes of DENV NS1activates the same pathways with other flaviviruses (Puerta-Guardo et al: Cell Reports, 26(6), 1598-1613, 2019). Cyclodextrins have been shown to possess broad-spectrum antiviral activity against human immunodeficiency virus (Khanna et al. J. Clin. Investig. 2002; 109:205-211.), herpes simplex virus, DENV (Jones et al., Sci. Adv. 2020; 6: eaax9318) and influenza virus (Goncharova et al. Acta Naturae. 2019 11(3): 20-30). These publications hypothesize mechanisms of action including inhibition of viral entry and replication, as well as virucidal activity through cholesterol-sequestering action.

Patent applications WO2018015465 and WO2009106986 raises the possibility of using cholesterol inhibitor molecules, including cyclodextrins (preferably methyl-beta-cyclodextrin), for modulating the activity of the Flavivirus NS1 protein, more specifically DENV NS1, by inhibiting NS1 secretion from infected cells. However, the document does not show data regarding exposure, therapeutic dose, mechanism of action, test methods, efficacy, or structure-activity relationship related to the effect of cyclodextrins and their derivatives on flavivirus infection or NS1-induced pathogenesis.

### BRIEF DESCRIPTION OF THE DRAWINGS

FIG. 1 shows the effect of 10 micrograms/mL of alpha-cyclodextrin sulfate sodium salt (degree of substitution ˜12), beta-cyclodextrin sulfate sodium salt (degree of substitution ˜13), and gamma-cyclodextrin sulfate sodium salt (degree of substitution ˜14) on endothelial dysfunction induced by DENV NS1.

FIG. 2 depicts the effect of 10 micrograms/mL 2,3-di-OH-6-sulfo-gamma cyclodextrin sodium salt (degree of substitution 8) on endothelial dysfunction induced by DENV NS1.

FIG. 3 depicts the effect of 1000 micrograms/mL sulfopropyl-alpha-cyclodextrin sodium salt (degree of substitution ˜2), sulfopropyl-beta-cyclodextrin sodium salt (degree of substitution ˜2), and sulfopropyl-gamma-cyclodextrin sodium salt (degree of substitution ˜2) on endothelial dysfunction induced by DENV NS1.

FIG. 4 shows the effect of 100 micrograms/mL 2,3-dimethyl-6-sulfate-beta cyclodextrin and 2,3-dimethyl-6-sulfate-gamma cyclodextrin on endothelial dysfunction induced by DENV NS1.

FIG. 5 shows the effect of 100 micrograms/mL of sulfobutylether-beta cyclodextrin of (degree of substitution ˜6.5, Dexolve®, Cyclolab) on endothelial dysfunction induced by DENV NS1.

FIG. 6 shows the effect of 1000 micrograms/mL of 2-hydroxypropyl beta cyclodextrin (degree of substitution: 4.6) and 2-hydroxypropyl gamma cyclodextrin (degree of substitution: 4.8) on endothelial dysfunction induced by DENV NS1.

### DETAILED DESCRIPTION

The present invention is based on the evaluation of the in vitro efficacy against DENV NS1-mediated pathogenesis of a series of different CDs that display variable cavity sizes as well as different degrees and types of substitution. In an in vitro model of endothelial permeability, CDs, at concentrations with no anticoagulant effects, were added to human pulmonary microvascular endothelial cells (HPMECs) treated with DENV NS1 to induce endothelial hyperpermeability.

Endothelial disruption (hyperpermeability) was quantified by measuring transendothelial electrical resistance (TEER). CDs bearing sulfate ester groups and sulfoalkyl moieties (at 10 and 100 micrograms/mL concentration, respectively) significantly reduced TEER values when compared to NS1-only treatment. No cytotoxicity was observed for any CD tested up to 1500 micrograms/mL. Sulfate group substitution of cyclodextrins contributed to the anti-leak activity.

Based on the above findings, we claim a new approach for the treatment and/or prevention of endothelial hyperpermeability leading to vascular leakage and/or increased virus dissemination upon flavivirus infection, which comprises administering an effective amount of a cyclodextrin or a pharmaceutically applicable salt thereof to a patient experiencing flavivirus infection.

### EXAMPLE 1

### Method to Assess the Effect of Cyclodextrins on Endothelial Dysfunction Induced by DENV NS1

The method is suitable to study the effect of NS1 from DENV from all serotypes and NS1 from other flaviviruses. HPMEC monolayers (6×10⁴ cells/transwell) cultured in transwell inserts for three days were treated with the CD compounds then treated or not with DENV NS1 at 10 micrograms/mL. Recombinant NS1 protein from DENV (Strain 16681) weas certified to be endotoxin-free and >95% purity (Native Antigen, United Kingdom). TEER was measured from 2 to 10 hours post-treatment. The area under the curve of the different treatments+DENV NS1 was compared by one-way ANOVA plus Dunnett's test to the DENV NS1-only control.

### EXAMPLE 2

### Effect of alpha cyclodextrin sulfate sodium salt on endothelial dysfunction induced by DENV NS1

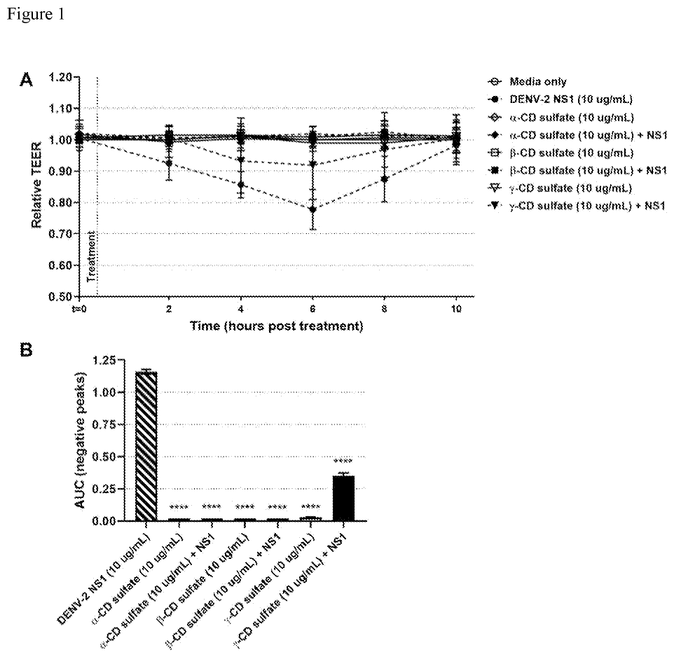

Randomly substituted alpha-cyclodextrin sulfate sodium salt (average degree of substitution: ˜12) was used as specified according to Example 1. FIG. 1 shows the effect of sulfated cyclodextrins (CDs) on endothelial dysfunction induced by DENV NS1. A) HPMEC monolayers (6×10e4 cells/transwell) cultured in transwell inserts for three days were treated with the compounds in the presence or absence of DENV NS1 at 10 ug/mL. TEER was measured from 2 to 10 hours post-treatment. Relative TEER was calculated as the ratio between resistance values obtained in the different treatments and medium-only controls. B) Area under the curve of the different treatments was compared by one-way ANOVA plus Dunnett's test to the DENV NS1control. P-values are summarized with number of asterisks as follows: *p≤0.05, **p≤0.01, ***p≤0.001, ****p≤0.0001. The effective dose of cyclodextrin for hindering endothelial leakage was 10 micrograms/mL.

### EXAMPLE 3

### Effect of Beta Cyclodextrin Sulfate Sodium Salt on Endothelial Dysfunction Induced by DENV NS1

Randomly substituted beta cyclodextrin sulfate sodium salt (average degree of substitution: ˜13) was used as specified according to Example 1. The results are shown in FIG. 1. The effective dose of cyclodextrin for hindering endothelial hyperpermeability was 10 micrograms/mL.

### EXAMPLE 4

### Effect of Gamma Cyclodextrin Sulfate Sodium Salt on Endothelial Dysfunction Induced by DENV NS1

Randomly substituted gamma cyclodextrin sulfate sodium salt (average degree of substitution: ˜14) was used as specified according to Example 1. The results are shown in FIG. 1. The effective dose of cyclodextrin for hindering endothelial leakage was 10 micrograms/mL.

### EXAMPLE 5

### Effect of 2,3-di-OH-6-Sulfo-Gamma Cyclodextrin Sodium salt on Endothelial Dysfunction Induced by DENV NS1

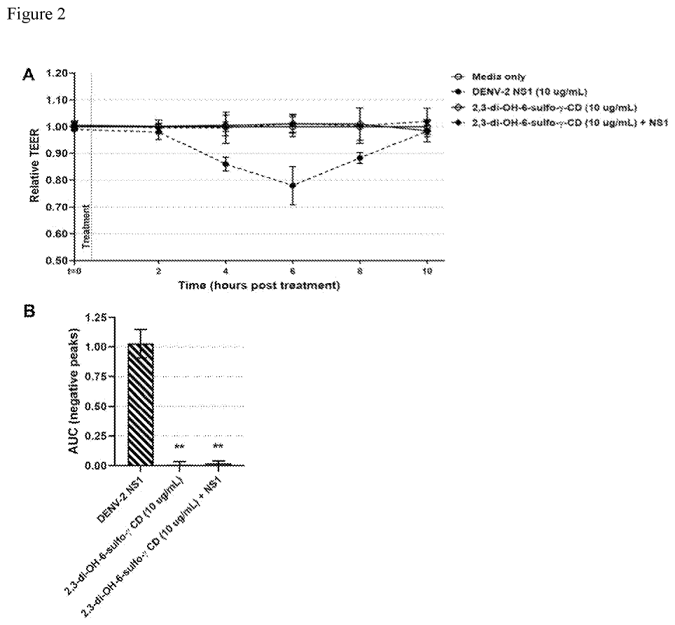

A single isomer of a gamma cyclodextrin sulfate sodium salt, the 2,3-di-OH-6-sulfo-gamma cyclodextrin sodium salt (degree of substitution: 8), was used as specified according to Example 1. FIG. 2 shows the effect of 2,3-di-OH-6-sulfo-gamma cyclodextrin sodium salt (2,3-di-OH-6-sulfo-γ CD) on endothelial dysfunction induced by DENV NS1. A) HPMEC monolayers (6×10⁴ cells/transwell) cultured in transwell inserts for 3 days were treated with the compound in the presence or absence of DENV NS1 at 10 ug/mL. TEER was measured from 2 to 10 hours post-treatment. Relative TEER was calculated as the ratio between resistance values obtained in the different treatment groups and medium-only control. B) Area under the curve of the different treatments were compared by one-way ANOVA+Dunnett's test to the DENV NS1 control. P-values are summarized with number of asterisks as follows: *p≤0.05, **p≤0.01, ***p≤0.001, ****p≤0.0001. The effective dose of cyclodextrin for inhibiting endothelial hyperpermeability was 10 micrograms/mL.

### EXAMPLE 6

### Effect of Sulfopropylated Alpha Cyclodextrin on Endothelial Dysfunction Induced by DENV NS1

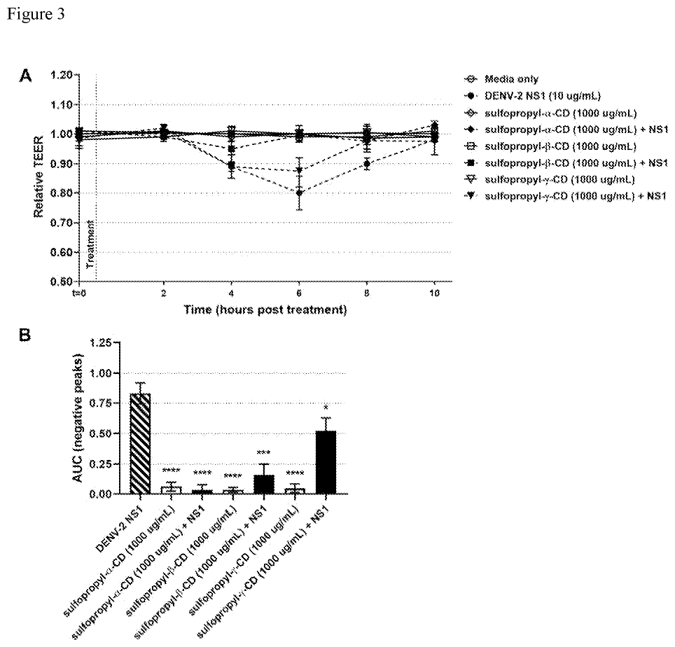

Randomly substituted sulfopropylether alpha cyclodextrin sodium salt (average degree of substitution: 2) was used as specified according to Example 1. FIG. 3 shows the effect of sulfopropyl-cyclodextrins on endothelial dysfunction induced by DENV NS1. A) HPMEC monolayers (6×10⁴ cells/transwell) cultured in transwell inserts for 3 days were treated with the compounds in the presence or absence of DENV NS1 at 10 ug/mL. TEER was measured from 2 to 10 hours post-treatment. Relative TEER was calculated as the ratio between resistance values obtained in the different treatments and medium-only controls. B) Area under the curve of the different treatments were compared by one-way ANOVA+Dunnett's test to the DENV NS1control. P-values are summarized with number of asterisks as follows: *p≤0.05, **p≤0.01, ***p≤0.001, ****p≤0.0001. The effective dose of cyclodextrin for hindering endothelial leakage was 1000 micrograms/mL.

### EXAMPLE 7

### Effect of Sulfopropylated Beta Cyclodextrin on Endothelial Dysfunction Induced by DENV NS1

Randomly substituted sulfopropylether beta cyclodextrin sodium salt (average degree of substitution: 2) was used as specified according to Example 1. The results are shown in FIG. 3. Effective dose of cyclodextrin for hindering endothelial dysfunction was 1000 micrograms/mL.

### EXAMPLE 8

### Effect of Sulfopropylated Gamma Cyclodextrin on Endothelial Dysfunction Induced by DENV NS1

Randomly substituted sulfopropylether gamma cyclodextrin sodium salt (average degree of substitution: 2) was used as specified according to Example 1. The results are shown in FIG. 3. The effective dose of cyclodextrin for hindering endothelial leakage was 1000 micrograms/mL.

### EXAMPLE 9

### Effect of Methylated Alfa Cyclodextrin Sulfate on Endothelial Dysfunction Induced by DENV NS1

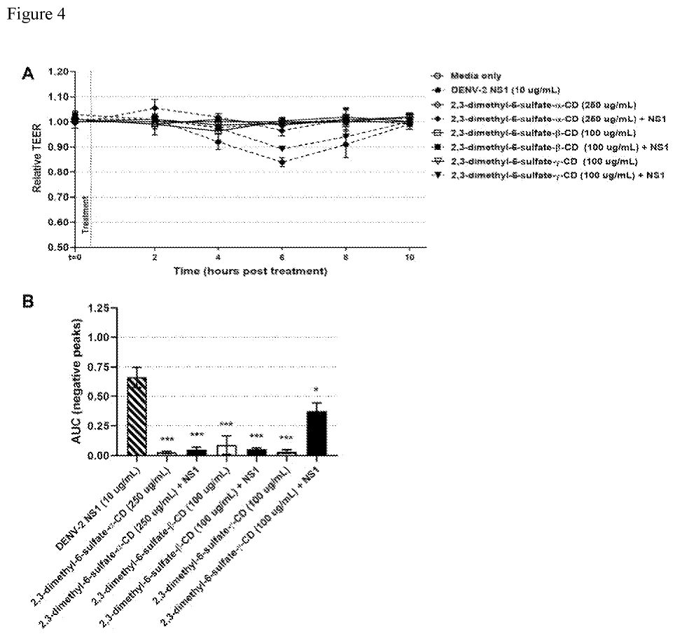

Single isomer 2,3-dimethyl-6-sulfate-alpha cyclodextrin sodium salt (degree of substitution: 6) used as specified according to Example 1. Effective dose of cyclodextrin for inhibiting endothelial hyperpermeability was 250 micrograms/mL. FIG. 4 shows the effect of 2,3-dimethyl-6-sulfate-alpha cyclodextrin on endothelial dysfunction induced by DENV NS1. A) HPMEC monolayers (6×10⁴ cells/transwell) cultured in transwell inserts for 3 days were treated with the compounds in the presence or absence of DENV NS1 at 10 ug/mL. TEER was measured from 2 to 10 hours post-treatment. Relative TEER was calculated as the ratio between resistance values obtained in the different treatments and medium-only controls. B) Area under the curve of the different treatments were compared by one-way ANOVA+Dunnett's test to the DENV NS1 control. P-values are summarized with number of asterisks as follows: *p≤0.05, **p≤0.01, ***p≤0.001, ****p≤0.0001.

### EXAMPLE 10

### Effect of Methylated Beta Cyclodextrin Sulfate on Endothelial Dysfunction Induced by DENV NS1

Single isomer 2,3-dimethyl-6-sulfate-beta cyclodextrin sodium salt (degree of substitution: 7) used as specified according to Example 1. The results are shown in FIG. 4. The effective dose of cyclodextrin for hindering endothelial leakage was 100 micrograms/mL.

### EXAMPLE 11

### Effect of Methylated Gamma Cyclodextrin Sulfate on Endothelial Dysfunction Induced by DENV NS1

Single isomer 2,3-dimethyl-6-sulfate-gamma cyclodextrin sodium salt (degree of substitution: 8) used as specified according to Example 1. The results are shown in FIG. 4. The effective dose of cyclodextrin for hindering endothelial leakage was 100 micrograms/mL.

### EXAMPLE 12

### Effect of Sulfobutylated Beta Cyclodextrin on Endothelial Dysfunction Induced by DENV NS1

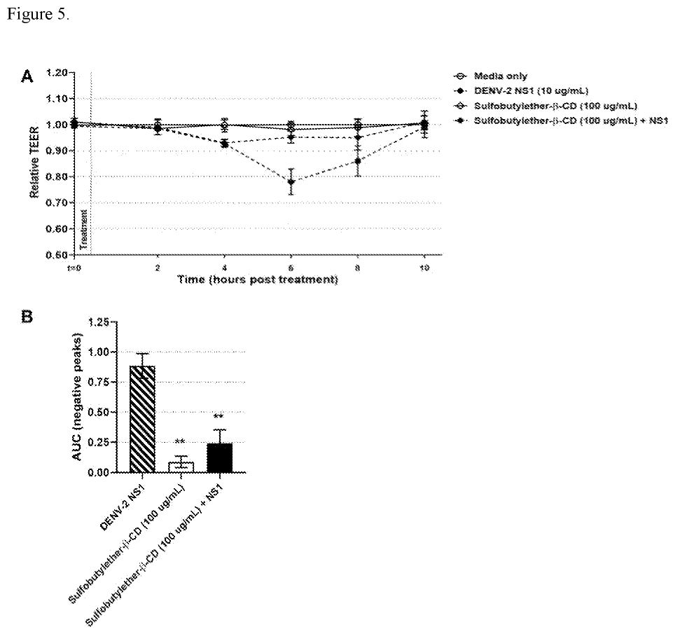

Randomly substituted sulfobutlyether beta cyclodextrin sodium salt (Dexolve®, Cyclolab, degree of substitution: 6.5) was used as specified according to Example 1. FIG. 5 shows the effect of sulfobutylether-beta-cyclodextrin on endothelial dysfunction induced by DENV NS1. A) HPMEC monolayers (6×10⁴ cells/transwell) cultured in transwell inserts for 3 days were treated with the compounds in the presence or absence of DENV NS1 at 10 ug/mL. TEER was measured from 2 to 10 hours post-treatment. Relative TEER was calculated as the ratio between resistance values obtained in the different treatments and media-only controls. B) Area under the curve of the different treatments were compared by one-way ANOVA+Dunnett's test to the DENV NS1 control. P-values are summarized with number of asterisks as follows: *p≤0.05, **p≤0.01, ***p≤0.001, ****p≤0.0001. The effective dose of cyclodextrin for hindering endothelial leakage was 100 micrograms/mL.

### EXAMPLE 13

### Effect of 2-Hydroxypropyl-Beta-Cyclodextrin on Endothelial Dysfunction Induced by DENV NS1

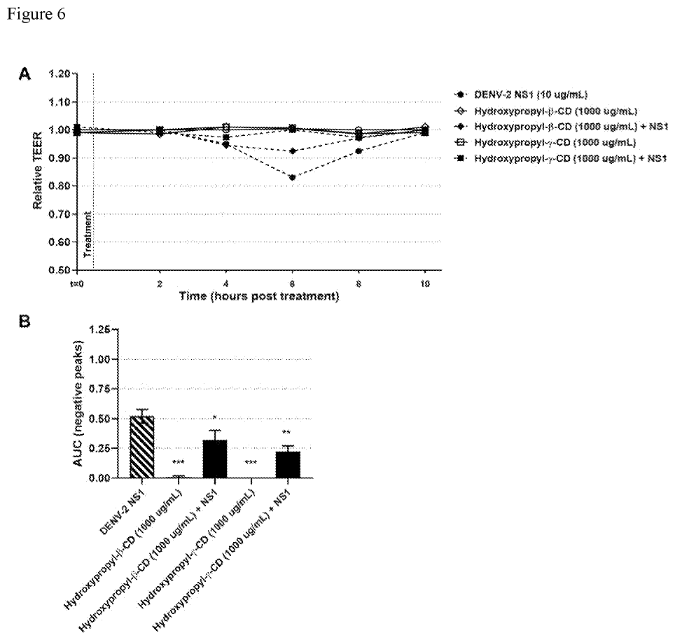

Randomly substituted 2-hydroxypropyl-beta-cyclodextrin (degree of substitution: 4.6) was used as specified according to Example 1. FIG. 6 shows the effect of 2-hydroxypropyl-beta-cyclodextrin on endothelial dysfunction induced by DENV NS1. A) HPMEC monolayers (6×10⁴ cells/transwell) cultured in transwell inserts for 3 days were treated with the compounds in the presence or absence of DENV NS1 at 10 ug/mL. TEER was measured from 2 to 10 hours post-treatment. Relative TEER was calculated as the ratio between resistance values obtained in the different treatments and medium-only controls. B) Area under the curve of the different treatments were compared by one-way ANOVA+Dunnett's test to the DENV NS1 control. P-values are summarized with number of asterisks as follows: *p≤0.05, **p≤0.01, ***p≤0.001, ****p≤0.0001. The effective dose of cyclodextrin for hindering endothelial leakage was 1000 micrograms/mL.

### EXAMPLE 14

### Effect of 2-Hydroxypropyl-Gamma-Cyclodextrin on Endothelial Dysfunction Induced by DENV NS1

Randomly substituted 2-hydroxypropyl-gamma-cyclodextrin (degree of substitution: 4.8) used as specified according to Example 1. The results are shown in FIG. 6. The effective dose of cyclodextrin for hindering endothelial leakage was 1000 micrograms/mL.

What is claimed is:

1. A method for the treatment or prevention of endothelial permeability condition followed by flavivirus infection leading to vascular leak and increased virus dissemination, which comprises administering an effective amount of a compound selected from at least one cyclodextrin, cyclodextrin derivative or a pharmaceutically applicable salt thereof to a patient in need thereof.

2. The method of claim 1, wherein at least one selected cyclodextrin is a cyclodextrin sulfate ester or a pharmaceutically applicable salt thereof.

3. The method of claim 2, wherein the cyclodextrin sulfate ester is alpha cyclodextrin sulfate ester or a pharmaceutically applicable salt thereof.

4. The method of claim 2, wherein the cyclodextrin sulfate ester is beta cyclodextrin sulfate ester or a pharmaceutically applicable salt thereof.

5. The method of claim 2, wherein the cyclodextrin sulfate ester is gamma cyclodextrin sulfate ester or a pharmaceutically applicable salt thereof.

6. The method of claim 3, wherein the cyclodextrin sulfate ester is sulfate ester of alpha cyclodextrin or a pharmaceutically applicable salt thereof further substituted with at least one alkyl, hydroxyalkyl or acetyl group.

7. The method of claim 4, wherein the cyclodextrin sulfate ester is sulfate ester of beta cyclodextrin or a pharmaceutically applicable salt thereof further substituted with at least one alkyl, hydroxyalkyl or acetyl group.

8. The method of claim 5, wherein the cyclodextrin sulfate ester is sulfate ester of gamma cyclodextrin or a pharmaceutically applicable salt thereof further substituted with at least one alkyl, hydroxyalkyl or acetyl group.

9. The method of claim 1, wherein at least one selected cyclodextrin is a sulfoalkylether cyclodextrin or a pharmaceutically applicable salt thereof.

10. The method of claim 9, wherein the cyclodextrin is sulfoalkylated alpha cyclodextrin or a pharmaceutically applicable salt thereof.

11. The method of claim 9, wherein the cyclodextrin is sulfoalkylated beta cyclodextrin or a pharmaceutically applicable salt thereof.

12. The method of claim 9, wherein the cyclodextrin is sulfoalkylated gamma cyclodextrin or a pharmaceutically applicable salt thereof.

13. The method of claim 10, wherein the sulfoalkylated alpha cyclodextrin or a pharmaceutically applicable salt thereof is further substituted with at least one alkyl, hydroxyalkyl or acetyl group.

14. The method of claim 11, wherein the sulfoalkylated beta cyclodextrin or a pharmaceutically applicable salt thereof is further substituted with at least one alkyl, hydroxyalkyl or acetyl group.

15. The method of claim 12, wherein the sulfoalkylated gamma cyclodextrin or a pharmaceutically applicable salt thereof is further substituted with at least one alkyl, hydroxyalkyl or acetyl group.

16. The method of claim 11, wherein the cyclodextrin is sulfobutylether beta cyclodextrin sodium salt.

17. The method of claim 1, wherein at least one selected cyclodextrin is 2-hydroxypropyl beta cyclodextrin or 2-hydroxypropyl gamma cyclodextrin.
